(19) United States
(12) Patent Application Publication
(10) Pub. No.: US 2007/0134787 A1
(43) Pub. Date: Jun. 14, 2007

(54) Living cell observing cell

(76) Inventors: Yoshiharu Shirakawabe, Chiba-shi (JP); Akira Inoue, Chiba-shi (JP); Naoya Watanabe, Chiba-shi (JP); Amiko Nihei, Chiba-shi (JP)

Correspondence Address: BRUCE L. ADAMS, ESQ.;SUITE 1231, 17 BATTERY PLACE, NEW YORK, NY, 10004, US

(21) Appl. No.: 11/583,197

(22) Filed: Oct. 19, 2006

(30) Foreign Application Priority Data
Oct. 25, 2005 (JP) 2005-309498

(51) Int. Cl. C12M 1/22; C12M 1/34

(52) U.S. Cl. 435/288.3; 435/288.7; 435/299.1; 435/305.1

(57) ABSTRACT

A living cell observing cell capable of measuring a surface of a membrane of a living cell or a rear surface side thereof and accurately performing structural analysis is provided. The living cell observing cell is used to culture at least one cell in a culture solution and observe the cells. The living cell observing cell includes a container body which stores the culturing solution and a flat location plate which is detachably fixed in the container body and has a plurality of protrusions formed in a predetermined interval on a surface thereof, wherein the cells are located on a plurality of the protrusions.

### BACKGROUND OF THE INVENTION

1. Field of the Invention

The present invention relates a living cell observing cell used to measure a living bio sample such as a culturing sample and a bio material in such as a field as a bio technology and a refurbished medical science.

More specifically, the invention relates to a living cell observing cell used for researches of performing structural analysis of function or expression mechanisms due to matrix measurement on uppermost surface of a cell, measuring activation of an ovum or the like, or measuring surface state at a cleavage period. In a cell membrane signalion research, the living cell observing cell can be used for research on stimuli to a particular or arbitrary portions.

Measurements of the cell activation or the ovum activation are expected to be a substitute for an animal test or a clinical test as applications thereof. Particularly, in case of ovum, measurement for screening of activation degree after artificial insemination is required. For the requirement, the living cell observing cell according to the invention is used as a tool for quantizing conventional screening which is performed by using naked eyes.

2. Description of Related Art

A convention culturing container utilizes glass or plastic charlet. The charlet is maintained in a desired environment (an environment required for growing a bio sample, referred to as a culturing environment), so that desired cells can be obtained. In addition, there is proposed a technique of adding a pressure or a temperature in order to grow the cells in a simulated environment similar to a living body environment. In addition, there is proposed a technique of preparing a plurality of vessels containing the cells and providing sensors to the vessels and performing electrical measurement for the vessels. These techniques are disclosed in JP-A-2005-192406 and JP-A-7-121232.

However, the conventional methods have the following problems.

In the measurement using a conventional culturing container or a container having measurement vessels (mainly, electrical measurement), the measurement is performed over a very wide region, so that a measurement interval is too wide.

In recent researches, very fine and specialized measurement is required for various applications, so that a very narrow measurement interval is needed. However, since there is no container coping with such requirements, the measurement cannot be performed.

In addition, there is no container capable of actively applying physical stimuli to living body sample and measuring a periphery of the living body sample in a fine narrow interval.

In general, in a case of culturing the cells, when the cells are inserted into a conventional charlet, filaments of the cells extend to spread over a bottom portion of the charlet, so that the cells are in surface-wise contact with the bottom portion of the charlet. For the reason, it is difficult to measure a lower portion of membrane, that is, a rear surface side of the cell and obtain information on the lower portion of membrane. In addition, although a large electrode is disposed at the lower portion of the charlet, the only measurement associated with the surface contact can be performed.

In addition, in order to uniformly dispose a probe on the surface of the cell, the probe is needed to be in contact with the cell along the shape of the cell with a uniform force. Therefore, it is difficult to control the probe in a narrow interval. In general, the surfaces of the cells exposed to the surface (adhering surface) of the cell fatty membranes which are in surface contact are different in terms of existing proteins and existence formation, and a research of measuring the adhering surface is made.

In the conventional case, the measurement is generally made on the upper portion of the culturing cell (surface of the cell), but it is practically difficult to measure the rear surface side of the cell. Although front or rear surfaces of proteins generally have different structures, since the measurement of the rear surface sides is difficult as described above, it is difficult to accurately perform structural analysis.

### SUMMARY OF THE INVENTION

The invention is to provide a living cell observing cell capable of measuring a surface of a membrane of a living cell or a rear surface side thereof and accurately performing structural analysis of the living cell.

In order to solve the aforementioned problems, the present invention provides the following aspects.

According to an aspect of the invention, there is provided a living cell observing cell used to culture at least one cells in a culturing solution and observe the cells, the living cell observing cell comprising: a container body which stores the culturing solution; and a flat location plate which is detachably fixed in the container body and has a plurality of protrusions formed in a predetermined interval on a surface thereof, wherein the cells are located on a plurality of the protrusions.

In the living cell observing cell, firstly, the location plate is fixed in the container body, and the culturing solution is stored in the container body. Therefore, the location plate is immersed into the culturing solution. Next, when the cells are sprayed into the culturing solution, the cells are located on a plurality of the protrusions formed on the surface of the location plate with a predetermined interval (for example, an interval of from hundreds nano-meters to several micro-meters) maintained between the cells. The cells are supported in a state where the cells are in a point-wise contact with a plurality of the protrusions. Therefore, unlike a conventional case where filaments of cells spread to be entirely in surface-wise contact with the bottom surface, the cells are in a state that the cells float within the culturing solution due to a plurality of the protrusions. Namely, the cells are in a state that the cells are located as naturally as in a living body.

Therefore, it is possible to measure the surface of the cell membrane or proteins at the rear sides of the cells which cannot easily measured conventionally. In addition, since the cells can be accurately observed in a more natural state, it is possible to perform structure analyzing at a high accuracy. As a result, it is possible to observe various aspects of the cells.

In addition, in the living cell observing cell, a plurality of the protrusions maybe be regularly aligned in an arrayed shape.

In the living cell observing cell, since a plurality of the protrusions are formed in an arrayed shape with a predetermined interval maintained, it is possible to more stably locate the cells.

In the living cell observing cell, a plurality of the protrusions may have a conductivity, and the living cell observing cell may include: a plurality of wire patterns which are formed on the location plate to be electrically connected to a plurality of the protrusions; a plurality of external connection wires which are formed on the location plate to be electrically connected to a plurality of the wire patterns and externally electrically connected; and an insulating film which is coated on the wire patterns and bottom sides of the protrusions in an electrically insulated state.

In the living cell observing cell, after the cells are located on a plurality of the protrusions, a predetermined voltage is applied across the cells and the protrusions selected among the protrusions contacting with the cells through the external connection wires and the wire patterns, so that currents flow from the selected protrusions into the cells. Therefore, stimuli are applied to the cells. At the same time, the currents and voltages flowing through the cells are measured by using other protrusions which are in point-wise contact with the cells. Therefore, since the current distribution or the like flowing through the cells can be measured, it is possible to perform signalion research on cell membrane by observing a network of the cells. As a result, it is possible to observe various aspects of cell.

In addition, since the bottom sides of the protrusions and the wire patterns are coated with the insulating film, the protrusions and the wire patterns cannot directly contacting with the culturing solution. Namely, since minimum regions corresponding to the distal ends of the protrusions are in contact with the culturing solution, the current cannot easily leak into the culturing solution, and the measurement can be accurately performed. In addition, stability can be improved.

In the living cell observing cell, a plurality of the protrusions may be made of a piezo-electric material and resistance values of the protrusions vary with forces exerted by the cells.

In the living cell observing cell, a plurality of the protrusions may be made of a piezo-electric material such as PZT (lead zirconate titanate), so that positions of the cells can be accurately specified by using resistance value of the protrusions contacting with the cells. Therefore, a time-varying state of the moving cells can be measured. As a result, it is possible to observe more various aspects of cell.

In the living cell observing cell, all the protrusions may be made of the same material.

In the living cell observing cell, since all the protrusions are made of the same material, a potential distribution can be measured. Namely, although non-uniform electrical field may be generated due to difference in protein density distribution or channels over the surfaces of the cell membranes, since all the protrusions are made of the same material, such a distribution can be measured.

In the living cell observing cell, a plurality of the protrusions may be made of different materials.

In the living cell observing cell, since a plurality of the protrusions are made of different materials, a potential gradient is generated between cell contact surfaces and the protrusions due to a difference between ionization tendencies thereof. At this time, since the protrusions are made of different materials, a potential gradient can be generated from known different metals, a weak potential can be applied to the cells in a self-aligned manner. As a result, matrix analysis can be formed by measuring signals for a change in potential applied to the protrusions.

In the living cell observing cell, the protrusions may have a height which is in a range of from 500 nm to 100 μm.

In the living cell observing cell, since the height of the protrusions are formed to be in a range of from 500 nm to 100 μm, the cells can be securely floated, so that it is possible to securely prevent lower portions of the cells at the rear side thereof from be in surface-wise contact with the surface of the location plate. As a result, measurement for the surface of cell membrane can be more accurately and securely performed.

In the living cell observing cell, the protrusions may have a height which is in a range of from 1 nm to 500 nm.

In the living cell observing cell, since the protrusions have a very small height in a range of from 1 nm to 500 nm, the located cells are in surface-wise contact with an upper surface of the location plate similar to a conventional one. Namely, a plurality of the protrusions are disposed on the bottom sides of the cells. As a result, unlike the conventional case, it is possible to accurately measure the rear surface side (bottom sides) of the cells.

In the living cell observing cell, the container body may include a lower mount on which the location plate is mounted and an upper mount which is detachably assembled with the lower mount to press the location plate and contain the culturing solution therein.

In the living cell observing cell, a location plate is mounted on the lower mount. Next, the upper mount is assembled into the lower mount. Therefore, the location plate is securely fixed in a state of pressing the lower mount. Next, the culturing solution is stored in the inner portion of the upper mount, so that the location plate can be immersed into the culturing solution. In particular, since the location plate is securely pressed on the lower mount by the upper mount, the location plate cannot float on the culturing solution but be maintained in a stable state. As a result, the cells can be stably located, and measurement accuracy can be improved.

According to the living cell observing cell of the invention, since the cells can be more accurately observed in a more natural state, it is possible to measure the surface of the cell membrane which is difficult in a conventional one and perform structure analyzing at a high accuracy.

### BRIEF DESCRIPTION OF THE DRAWINGS

The invention will be described with reference to the accompanying drawings, wherein like numbers reference like elements.

FIG. 1 is a constructional view showing a living cell observing cell according to a n embodiment of the invention.

FIG. 2 is a top view of a location plate constituting the living cell observing cell shown in FIG. 1.

FIG. 3 is an enlarged cross sectional view of the location plate shown in FIG. 2.

FIG. 4 is a cross sectional view of an SOI substrate, that is, a staring substrate as a view showing a manufacturing process for the location plate shown in FIG. 3.

FIG. 5 is a cross sectional view of the SOI substrate showing a state where a photoresist film is patterned on a silicon activation layer of the SOI substrate shown in FIG. 4 as a view showing-a manufacturing process for the location plate shown in FIG. 3.

FIG. 6 is a cross sectional view showing a state where a plurality of protrusions are formed by etching the silicon activation layer using the photoresist layer as a mask in the state shown in FIG. 5 as a view showing a manufacturing process for the location plate shown in FIG. 3.

FIG. 7 is a cross sectional view showing a state where a metal film is coated on the silicon activation film including a plurality of the protrusions in the state shown in FIG. 6 as a view showing a manufacturing process for the location plate shown in FIG. 3.

FIG. 8 is a cross sectional view showing a state where a photoresist is patterned on the metal film in the state shown in FIG. 7 as a view showing a manufacturing process for the location plate shown in FIG. 3.

FIG. 9 is a cross sectional view showing a state where a wire pattern is formed by etching the metal film using the photoresist layer as a mask in the state shown in FIG. 8 as a view showing a manufacturing process for the location plate shown in FIG. 3.

FIG. 10 is a cross sectional view showing a state where the photoresist film is removed from the state shown in FIG. 9 as a view showing a manufacturing process for the location plate shown in FIG. 3.

FIG. 11 is a cross sectional view showing a state where an insulating film is coated on the silicon activation layer including the wire pattern and the protrusions in the state shown in FIG. 10 as a view showing a manufacturing process for the location plate shown in FIG. 3.

FIG. 12 is a cross sectional view showing a state where the insulating film is etched by a predetermined thickness thereof and distal ends of the protrusions coated with the metal film in the state shown in FIG. 11 as a view showing a manufacturing process for the location plate shown in FIG. 3.

### REFERENCE NUMERALS

S: cell
W: culturing solution
1: living cell culturing cell
2: container body
3 protrusion
4: location plate
11: upper mount
10: lower mount
25: wire pattern
26: external connection wire
27: insulating film

### DETAILED DESCRIPTION OF PREFERRED EMBODIMENTS

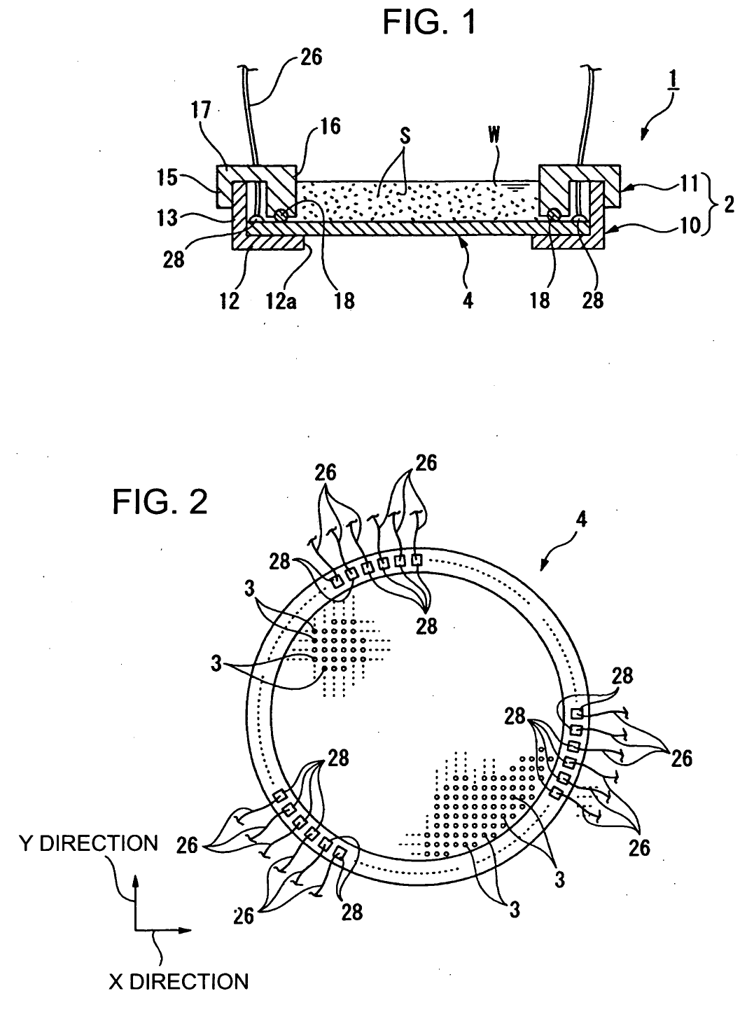

Hereinafter, a living cell observing cell according to an embodiment of the invention will be described with reference with FIGS. 1 to 12.

The living cell observing cell 1 is used to culture at least one cell S in a culture solution W and observe the cells S. As shown in FIG. 1, the living cell observing cell 1 includes a container body 2 which stores the culturing solution W and a flat location plate 4 which is detachably fixed in the container body 2 and has a plurality of protrusions 3 formed in a predetermined interval on a surface thereof, wherein the cells S are located on a plurality of the protrusions 3.

The container body 2 includes a lower mount 10 on which the location plate 4 is mounted and an upper mount 11 which is detachably assembled with the lower mount 10 to press the mounted location plate 4 and contain the culturing solution W therein.

The lower mount 10 includes a bottom plate 12 having an opening 12a at a central portion thereof and a wall 13 which is integrally formed together with the bottom plate 12 to be curved at an angle of about 90° from a circumference of the bottom plate 12. The location plate 4 is mounted on an upper surface of the bottom plate 12.

The upper mount 11 is integrally formed in a shape of ring with an outer wall 15, an inner wall 16, and an upper plate 17 which connects the outer wall 15 to the inner wall 16. An inner surface of the outer wall 15 of the upper mount 11 is slidingly inserted into an outer surface of the wall 13 of the lower mount 10. As a result, the upper and lower mounts 11 and 10 are assembled to be fixed to each other.

An O-ring 18 is attached on a lower surface of the inner wall 16 of the upper mount 11. When the two mounts 10 and 11 are assembled, the location plate 4 is arranged to press the lower mount 10 through the O-ring 18. As a result, the location plate 4 is maintained in a stable state without position deviation thereof.

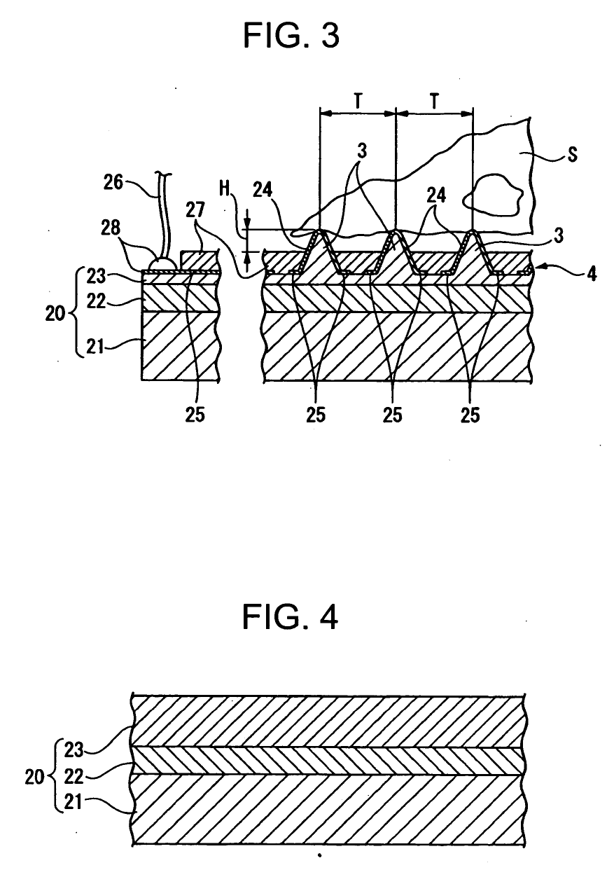

As shown in FIGS. 2 and 3, the location plate 4 is formed by using an SOI (Silicon On insulator) substrate 20 constructed by thermally attaching a silicon supporting layer 21, a silicon oxide layer 22, and a silicon activation layer 23 in a shape of circle (as seen from a top surface) so as to be inserted into the wall 13 of the wall 13 of the lower mount 10. A method of manufacturing the location plate 4 is described in detail later.

In addition, the protrusions 3 according to the embodiment is coated with a metal film 24 so as to have a conductivity. The location plate 4 includes a plurality of wire patterns 25 which to be electrically connected to a plurality of the protrusions 3, a plurality of external connection wires 26 which are electrically connected to the wire patterns and externally eclectically connected, and an insulating film 27 which is coated on the wire patterns 25 and the bottom sides of the protrusions 3 in an electrically insulated state.

All the protrusions 3 are made of the same material as the silicon activation layer 23, and, as shown in FIG. 2, regularly aligned in an arrayed shape in X and Y directions. Here, as shown in FIG. 3, the interval T of the protrusions 3 are adjusted to be in a range from hundreds of nano-meters to several micro-meters. In addition, a length H of the protrusions 3 protruding upward from the insulating film 27 is adjusted to be in a range of 500 nm to 100 μm.

The distal ends of the protrusions 3 are formed in a rounded shaped with a radius of curvature of from several nano-meters to hundreds nano-meters. The protrusions 3 are eclectically connected to the wire patterns 25 at a lower side of the insulating film 27.

The wire patterns 25 extend to a circumference of the location plate 4 to be electrically connected to electrode pads 28 which are formed along the circumference. As shown in FIG. 1, the electrode pads 28 are formed so as to be disposed between the O-ring 18 of the upper mount 11 and the wall 13 of the lower mount 10. As a result, it is possible to prevent the electrode pads 28 from directly contacting with the culturing solution W.

One end of each external connection wire 26 is connected to each electrode pad 28. The other end of each external connection wire 26 is connected to a voltage applying unit (not shown).

Hereinafter, a method of manufacturing the aforementioned location plate 4 is described.

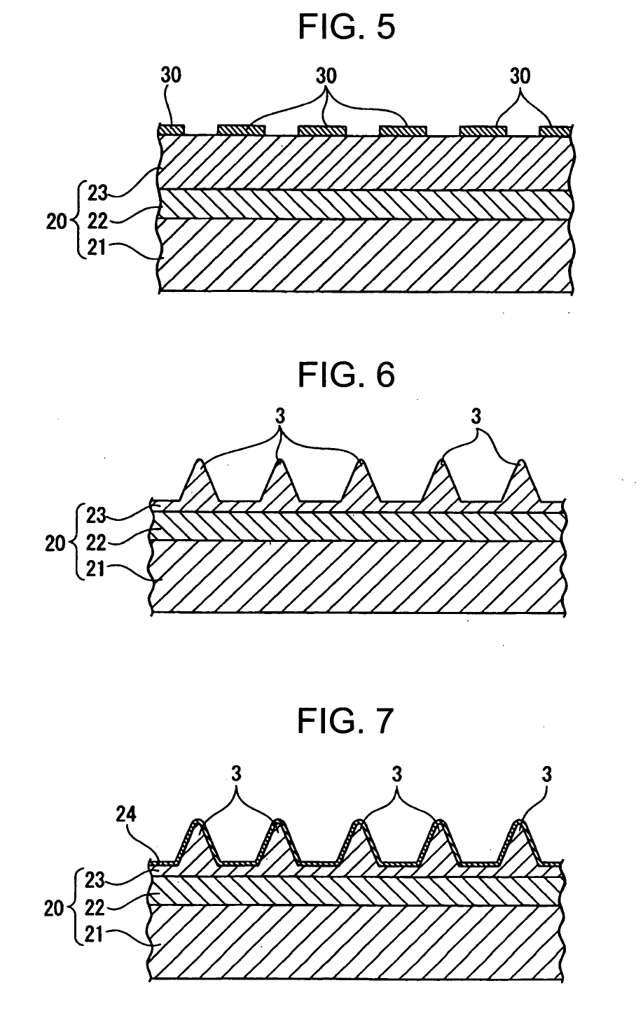

Firstly, a photoresist film 30 is formed as a etch mask on the silicon activation layer 23 of the SOI substrate 20 shown in FIG. 4. As shown in FIG. 5, the photoresist film 30 is patterned in a shape of dots at positions where the protrusions 3 are formed by using a photoresist technique. Namely, the photoresist films 30 are patterned to be regularly aligned in an arrayed shape in X and Y directions.

Next, as shown in FIG. 6, an RIE (Reactive Ion Etching) or DRIE (Deep Reactive Ion Etching) process is performed by using the photoresist film 30 as a mask to selectively remove the non-masked portions of the silicon activation layer 23 and form a plurality of the protrusions 3.

At this time, a reaction speed or the like is adjusted so as for the height of the protrusions 3 to be a predetermined height by taking into consideration a thickness of the insulating film 27 described later. After the formation of the protrusions 3 is completed, the photoresist film 30 used as a mask is removed.

Next, as shown in FIG. 7, a metal sputtering process is performed to coat the metal film 24 on the entire surface of the silicon activation layer 23 including the protrusions 3. As a result, the protrusions 3 are in a conductive state and are electrically connected to each other.

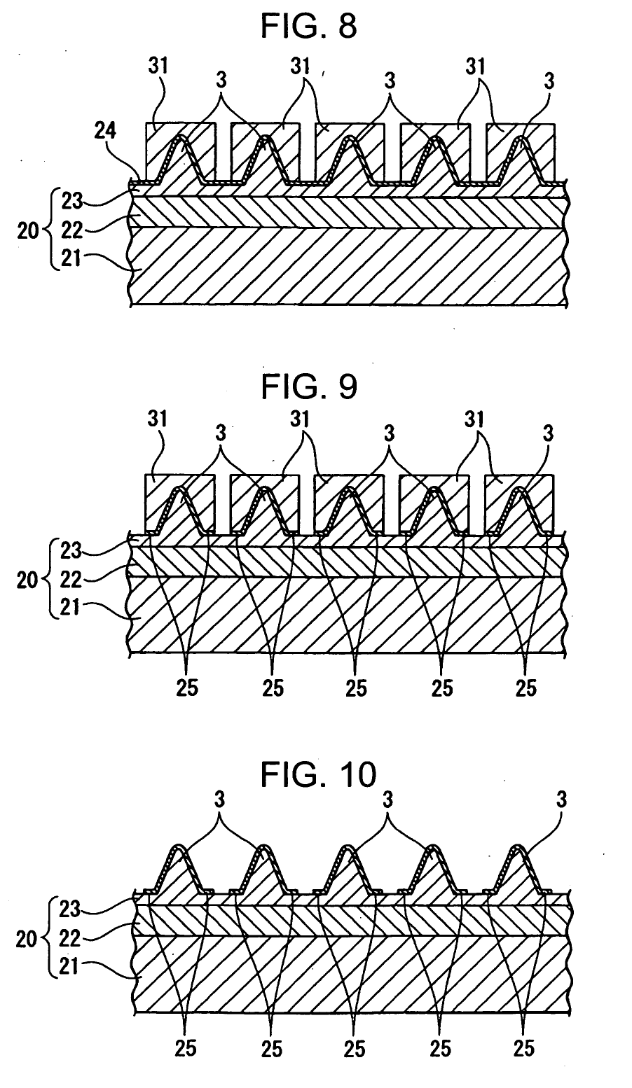

As shown in FIG. 8, a photoresist film 31 is patterned to cover regions excluding regions where the wire patterns 25 are formed. Distal ends of the protrusions 3 are temporarily protected by the photoresist film 31.

Next, as shown in FIG. 9, the metal film 24 is patterned by using the photoresist film 31 as a mask. Next, the photoresist film 31 is removed, so that the wire patterns 25 are electrically connected to the protrusions 3 as shown in FIG. 10. At the same time, an etching process is performed on the wire patterns 25 at the circumference of the silicon activation layer 23 to form the electrode pads 28.

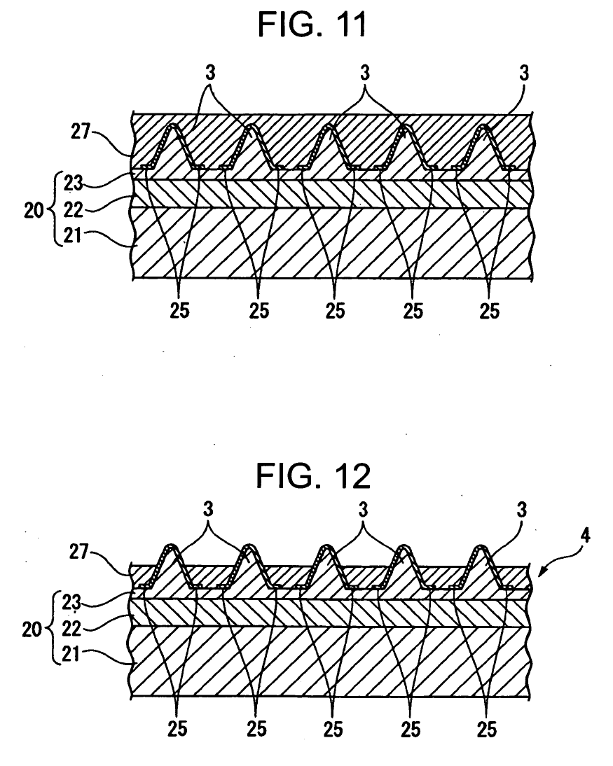

Next, as shown in FIG. 11, the insulating film 27 is coated on the silicon activation layer 23. Next, as shown in FIG. 12, an etching process is performed on the insulating film 27 to have a predetermined thickness. As a result, as shown in FIG. 1, the location plate 4 where a plurality of the protrusions 3 having a height ranging from 500 nm to 100 μm are formed to protrude from the insulating film 27 can be manufactured.

Hereinafter, observation of cells S in the culturing solution W contained in the aforementioned living cell observing cell is described.

Firstly, the living cell observing cell 1 is assembled. More specifically, the location plate 4 is mounted on the bottom plate of the lower mount 10. Next, the inner surface of the outer wall 15 of the upper mount 11 is slidingly assembled into the outer surface of the wall 13 of the lower mount 10, so that the lower mount 10 is engaged with the upper mount 11. Therefore, the location plate 4 is securely fixed in a state of pressing the lower mount 10 through the O-ring 18. In addition, the electrode pads 28 of the location plate 4 and the external connection wires 26 are disposed between the O-ring 18 and the wall 13 of the lower mount 10.

Next, the culturing solution W is stored in the inner portion of the upper mount 11. Therefore, the location plate 4 is in a state that the location plate 4 is immersed into the culturing solution W. As described above, since the electrode pads 28 and the external connection wires 26 are disposed outside the O-ring 18, it is possible to prevent the electrode pads 28 and the external connection wires 26 from directly contacting with the culturing solution W. In addition, since the location plate 4 is pressed on the lower mount 10 by the upper mount 11, the location plate 4 cannot float on the culturing solution W but be maintained in a stable state.

Next, the cells S are injected into the culturing solution W. The injected cells S moves through the culturing solution W to approach the location plate 4 so as to be located on a plurality of the protrusions 3 formed on the surface of the location plate 4 as shown in FIG. 3. Namely, the cells S are supported in a state where the cells S are in a point-wise contact with a plurality of the protrusions 3 in the culturing solution W. Therefore, unlike a conventional case where filaments of cells S spread to be entirely in surface-wise contact with the bottom surface, the cells S are in a state that the cells float within the culturing solution due to a plurality of the protrusions 3. Namely, the cells S are in a state that the cells are located as naturally as in a living body.

Particularly, since the height H of the protrusions 3 are formed to be in a range of from 500 nm to 100 μm, the cells S can be securely floated, so that it is possible to securely prevent lower portions of the cells at the rear side thereof from be in surface-wise contact with the surface of the location plate 4.

Therefore, since the cell membrane has the same property as the surface, it is possible to measure the surface of the cell membrane in a natural state which cannot easily measured due to deviation or functional protein existence formation between an exposed surface (surface of cell) and an rear surface (adhering surface) in a conventional one. In addition, since the cells S can be accurately observed in a more natural state, it is possible to perform structure analyzing at a high accuracy. Particularly, since the location plate 4 cannot float on the culturing solution W but be maintained in a stable state due to the upper and lower mounts 11 and 10, it is possible to stably locate the cells S. As a result, it is possible to improve measurement accuracy.

In an example of measurement, after the cells S are located, a predetermined voltage is applied across the cells S and the protrusions selected among the protrusions 3 contacting with the cells S through the external connection wires 26 and the wire patterns 25, so that currents flow from the protrusions 3 into the cells S. Therefore, stimuli are applied to the cells S. At the same time, the currents and voltages flowing through the cells S are measured by using other protrusions which are in point-wise contact with the cells S. Therefore, since the current distribution or the like flowing through the cells S can be measured, it is possible to perform signalion research on cell membrane by observing a network of the cells S. As a result, it is possible to observe various aspects of cell.

In addition, since the bottom sides of the protrusions 3 and the wire patterns 25 are coated with the insulating film 27, the protrusions 3 and the wire patterns 25 cannot directly contacting with the culturing solution W. Namely, since minimum regions corresponding to the distal ends of the protrusions 3 are in contact with the culturing solution W, the current cannot easily leak into the culturing solution W. Therefore, the measurement can be accurately performed, and stability can be improved.

In addition, since all the protrusions 3 are made of the same material, the potential distribution or the like of the cells S can be measured. Namely, although non-uniform electrical field may be generated due to difference in protein density distribution or channels over the surfaces of the cell membranes, since all the protrusions 3 are made of the same material, such a distribution can be measured. As a result, it is possible to observe more various aspects of cell.

Hereinbefore, although the preferred embodiment is described, the invention is not limited to the embodiment, but various modifications can be made without departing from a spirit and scope of the invention.

In the above-described embodiment, the height H of the protrusions 3 are formed to be in a range of from 500 nm to 100 μm, but not limited thereto. For example, a very small height in a range of from 1 nm to 500 nm may be formed. By doing so, the cells S located on the protrusions 3 may be in surface-wise contact with the surface of the location plate 4 similar to the conventional case in terms of outer appearance. Namely, a plurality of the protrusions 3 may be disposed on the bottom sides of the cells S. As a result, unlike the conventional case, it is possible to accurately measure the rear surface side (bottom sides) of the cells S.

In addition, a plurality of the protrusions 3 may be made of a piezo-electric material such as PZT (lead zirconate titanate) so that resistance value of the protrusions 3 can be varied with forces exerted by the cells S. Therefore, it is possible to accurately specify positions of the cells S by monitoring the resistance values of the protrusions 3 contacting with the cells S. Therefore, a time-varying state of the moving cells S can be measured. As a result, it is possible to observe more various aspects of cell.

In addition, although the location plate 4 is manufactured by using the SOI substrate 20, the invention is not limited to the SOI substrate 20. In addition, although all the protrusions 3 are made of the same material as that of the silicon activation layer 23, the protrusions may be made of different materials.

By doing so, a potential gradient is generated between the cell contact surfaces and the protrusions 3 due to a difference between ionization tendencies thereof. At this time, since the protrusions 3 are made of different materials, a potential gradient can be generated from known different metals, a weak potential can be applied to the cells S in a self-aligned manner. As a result, matrix analysis can be formed by measuring signals for a change in potential applied to the protrusions 3.

In addition, in the embodiment, the location plate 4 is fixed by using the container body 2 including the lower and upper mounts 10 and 11, but the invention is not limited thereto. A conventional culturing dish or charlet may be used as a container body, and a living cell observing cell may be constructed by inserting the location plate 4 into the container body.

In addition, in the embodiment, the protrusions 3 are formed to have a conductivity by coating the metal film 24 thereon, but the invention is not limited thereto. For example, the protrusions 3 may be formed to have a conductivity by doping impurities into the silicon activation layer 23 using an ion injection method.

What is claimed is:

1. A living cell observing cell used to culture at least one cells in a culturing solution and observe the cells, the living cell observing cell comprising:
a container body which stores the culturing solution; and
a flat location plate which is detachably fixed in the container body and has a plurality of protrusions formed in a predetermined interval on a surface thereof, wherein the cells are located on a plurality of the protrusions.

2. The living cell observing cell according to claim 1, wherein a plurality of the protrusions are regularly aligned in an arrayed shape.

3. The living cell observing cell according to claim 1, wherein a plurality of the protrusions has a conductivity, and wherein the living cell observing cell further comprises:
a plurality of wire patterns which are formed on the location plate to be electrically connected to a plurality of the protrusions;
a plurality of external connection wires which are formed on the location plate to be electrically connected to a plurality of the wire patterns and externally electrically connected; and
an insulating film which is coated on the wire patterns and bottom sides of the protrusions in an electrically insulated state.

4. The living cell observing cell according to claim 3, wherein a plurality of the protrusions are made of a piezo-electric material and resistance values of the protrusions vary with forces exerted by the cells.

5. The living cell observing cell according to claim 3, wherein all the protrusions are made of the same material.

6. The living cell observing cell according to claim 3, wherein a plurality of the protrusions are made of different materials.

7. The living cell observing cell according to claim 1, wherein the protrusions has a height which is in a range of from 500 nm to 100 μm.

8. The living cell observing cell according to claim 1, wherein the protrusions has a height which is in a range of from 1 nm to 500 nm.

9. The living cell observing cell according to claim 1, wherein the container body comprises a lower mount on which the location plate is mounted and an upper mount which is detachably assembled with the lower mount to press the location plate and contain the culturing solution therein.

10. The living cell observing cell according to claim 2, wherein a plurality of the protrusions has a conductivity, and wherein the living cell observing cell further comprises:
a plurality of wire patterns which are formed on the location plate to be electrically connected to a plurality of the protrusions;
a plurality of external connection wires which are formed on the location plate to be electrically connected to a plurality of the wire patterns and externally electrically connected; and
an insulating film which is coated on the wire patterns and bottom sides of the protrusions in an electrically insulated state.

11. The living cell observing cell according to claim 10, wherein a plurality of the protrusions are made of a piezo-electric material and resistance values of the protrusions vary with forces exerted by the cells.

12. The living cell observing cell according to claim 10, wherein all the protrusions are made of the same material.

13. The living cell observing cell according to claim 10, wherein a plurality of the protrusions are made of different materials.

14. The living cell observing cell according to claim 2, wherein the protrusions has a height which is in a range of from 500 nm to 100 μm.

15. The living cell observing cell according to claim 2, wherein the protrusions has a height which is in a range of from 1 nm to 500 nm.

16. The living cell observing cell according to claim 2, wherein the container body comprises a lower mount on which the location plate is mounted and an upper mount which is detachably assembled with the lower mount to press the location plate and contain the culturing solution therein.
